(19) United States
(12) Patent Application Publication
(10) Pub. No.: US 2024/0335326 A1
(43) Pub. Date: Oct. 10, 2024

(54) DEVICE IN THE FORM OF A BANDAGE PATCH WITH INGREDIENTS FOR BUG BITE RELIEF

(71) Applicant: Mor Ventures Inc., San Francisco, CA (US)

(72) Inventors: Ilyssa NORDA, San Francisco, CA (US); Diana RYDBECK, Coral Gables, FL (US); Paru CHAUDHARI, Pleasant Hill, CA (US)

(73) Assignee: Mor Ventures Inc., San Francisco, CA (US)

(21) Appl. No.: 18/626,694

(22) Filed: Apr. 4, 2024

Related U.S. Application Data
(60) Provisional application No. 63/495,000, filed on Apr. 7, 2023.

(51) Int. Cl. A61F 13/00; A61F 13/0203

(52) U.S. Cl. CPC A61F 13/00063; A61F 13/0213

(57) ABSTRACT

Disclosed herein are devices and methods for treating bug bites. In one variation, a treatment patch for use on a patient's skin can be provided, comprising a first surface comprising a backing sheet; a second surface comprising a hydrocolloid layer, wherein the hydrocolloid layer comprises a self-adhesive dressing configured to adhere the treatment patch to the patient at the second surface, wherein the hydrocolloid layer comprises aloe barbadensis leaf and witch hazel embedded within; and wherein the treatment patch comprises an ultraviolet protection factor (UPF) to improve scarring of the skin.

### CROSS-REFERENCE TO RELATED APPLICATION

This present application claims priority to U.S. Provisional Application No. 63/495,000, filed Apr. 7, 2023, which is incorporated by reference herein in its entirety.

### FIELD OF THE INVENTION

The present disclosure relates to systems for providing relief to a bug bite with a patch comprising a hydrocolloid layer embedded with witch hazel and aloe vera.

### BACKGROUND OF THE INVENTION

Insect bites can cause discomfort and skin irritation due to scratching and itching. The physiological roles of itching are increasingly understood and appreciated. The pathophysiological consequences of itching impact quality of life as much as pain. These dynamics have led to increasingly deep dives into the mechanisms that underlie and contribute to the sensation of itching.

Barrier damage, dry skin and itch are intricately linked and form the basis of many common skin diseases. Damage from environmental insults, or genetic or inflammatory causes, can impair the skin barrier, resulting in an increase in trans epidermal water loss and activation of itch-associated nerve fibers. The itch-scratch cycle can perpetuate skin barrier damage and itch. Topical therapeutic strategies are utilized to overcome dry skin and itch, primarily in the form of emollients. Recent advances in the understanding of the mechanisms underlying itch have enabled the development of new topical therapies, which may be incorporated into existing treatment regimes. Ultimately, treatment of dry skin and itch must be highly tailored to the individual according to their needs.

Current insect repellants such as sprays or creams can leak in containers, can contain toxic side effects, and can be abraded or washed off in environmental conditions.

Therefore, there remains a need for a flexible solution to the itch-scratch cycle that prevents both itching and scratching and that is wearable in all environmental conditions.

### SUMMARY

In one variation, a treatment patch for use on a patient's skin can be provided, comprising a first surface comprising a backing sheet; a second surface comprising a hydrocolloid layer, wherein the hydrocolloid layer comprises a self-adhesive dressing configured to adhere the treatment patch to the patient at the second surface, wherein the hydrocolloid layer comprises aloe barbadensis leaf and witch hazel embedded within; and wherein the treatment patch comprises an ultraviolet protection factor (UPF) to improve scarring of the skin.

The hydrocolloid layer can comprise a thickness of about 12 mm to about 18 mm. The backing sheet can be made of polyurethane and can be transparent or opaque. The treatment patch can be configured to adhere to the skin for 5 days. The hydrocolloid layer can comprise menthol, capsaicin, calendula, and/or hydrocortisone. The witch hazel can be diluted in water. The aloe barbadensis leaf can be purified and chopped into powder. The hydrocolloid layer can comprise zinc oxide.

Additionally, a method of using a treatment patch to relieve a region on a patient's skin can be provided. The method can comprise: diluting witch hazel in a water solution; purifying aloe barbadensis leaf and chopping the aloe barbadensis leaf into a powder; embedding the witch hazel and the aloe barbadensis leaf into a hydrocolloid layer; providing a treatment patch, wherein the treatment patch comprises: a first surface comprising a backing sheet, a second surface comprising the hydrocolloid layer, wherein the hydrocolloid layer comprises a self-adhesive dressing configured to adhere the treatment patch to the patient at the second surface, and wherein the treatment patch comprises an ultraviolet protection factor (UPF) to improve scarring of the skin; and administering the treatment patch to the patient's skin.

### BRIEF DESCRIPTION OF THE DRAWINGS

FIG. 1 illustrates a top view of a treatment patch in accordance with one variation of the invention.

FIG. 2 illustrates a side view of a treatment patch in accordance with one variation of the invention.

FIG. 3 illustrates a top view of one or more patches on a cold seal paper.

FIG. 4 illustrates a side view of the one or more patches between two cold seal papers.

FIGS. 5 and 6 illustrate further variations of one or more patches on a cold seal paper.

### DETAILED DESCRIPTION OF THE INVENTION

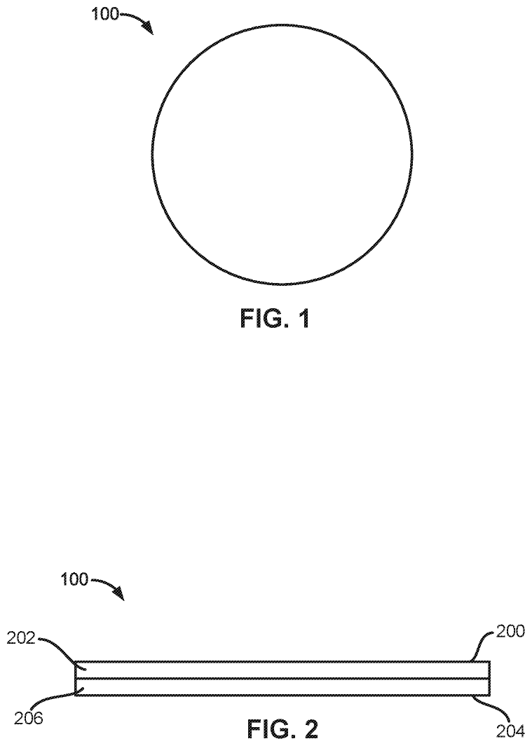

FIG. 1 is a schematic representation of a treatment patch 100 in accordance with one variation of the invention. The treatment patch 100 can comprise a generic circle shape to conform with a wide range of bug bite scars. Alternatively, the treatment patch 100 can be tailored to various skin conditions and as such can be round, oval, square, large, small, and other shapes or sizes.

FIG. 2 illustrates a side view of the treatment patch 100. The treatment patch 100 can comprise a first surface 200 comprising a backing sheet 202 and a second surface 204 comprising a hydrocolloid layer 206. The backing sheet 202 can be made of polyurethane. Alternatively, the backing sheet 202 can comprise acrylic sealants, silicone sealants, polylactic acid (PLA), or a combination thereof.

The hydrocolloid layer 206 can be a moisture-retentive dressing which contains gel-forming agents such as sodium carboxymethylcellulose and gelatin. The gel-forming properties with elastomers and adhesives can be applied to a carrier such as foam or film to form an absorbent, self-adhesive, waterproof wafer. In the presence of wound exudate, the hydrocolloid layer 206 can absorb liquid and form a gel, the properties of which are determined by the nature of the formulation. In sheet form, the layer can be either semi-occlusive or occlusive. Hydrocolloid interaction can debride by autolysis and can reduce dressing frequency depending on amount of exudate and the type of the hydrocolloid used.

The hydrocolloid layer 206 can comprise an active surface coated with a cross-linked adhesive mass containing a dispersion of gelatin, pectin and carboxymethyl cellulose together with other polymers and adhesives forming a flexible wafer. In contact with wound exudate, the polymers can absorb water and swell, forming a gel. The gel can be designed to drain, or to remain within the structure of the adhesive matrix which adheres to the patient's skin during administration of the path 100.

The hydrocolloid layer 206 can provide wound healing and can protect the top layer of the patient's skin. The application of self-adhesive hydrocolloid dressings, most commonly associated with the treatment of ulcerative conditions such as pressure ulcers and leg ulcers, can also offer in the management of acute wounds of all types, for example, decreasing healing times of donor sites by about 40% compared with traditional treatments. Healing times of superficial traumatic injuries and surgical wounds can be similarly enhanced.

The hydrocolloid layer 206 can comprise aloe barbadensis leaf (i.e., aloe vera) and witch hazel embedded within in order to improve itch and inflammation as a result of bug bites on the skin.

Aloe vera can inhibit the cyclooxygenase pathway and reduces prostaglandin E2 production from arachidonic acid. During manufacturing, the aloe barbadensis leaf can purified and chopped into powder before embedding into the hydrocolloid layer 206.

Glucomannan, a mannose-rich polysaccharide, and gibberellin, a growth hormone, interacts with growth factor receptors on the fibroblast, thereby stimulating its activity and proliferation, which in turn significantly increases collagen synthesis after topical and oral aloe vera. Aloe gel increases collagen content of the wound but also changes collagen composition (more type III) and increases the degree of collagen cross linking. This can result in accelerated wound contraction and increased the breaking strength of resulting scar tissue. Such treatment can also increase synthesis of hyaluronic acid and dermatan sulfate in the granulation tissue of a healing wound.

Witch hazel leaves and bark can contain up to 10% tannins, which contribute to astringent properties. Bark can contain mostly hamamelitannins and catechols while the leaves can contain more proanthocyanidins, ellagitannins and some essential oils. Tannins and polyphenols such as gallic acid are strong antioxidants and can have a protective effect on fibroblast cells against H₂O₂ induced damage. During manufacturing, witch hazel can be diluted in a water solution before embedding into the hydrocolloid layer.

The hydrocolloid layer 206 can further comprise menthol, capsaicin, calendula, hydrocortisone, or any combination thereof embedded within.

Menthol is selective activator of transient receptor potential melastatin-8 (TRPM8) channels and can also be a vasoactive compound. As a topical agent, menthol can act as a counter-irritant by imparting a cooling effect and by initially stimulating nociceptors and then desensitizing them. Topically applied menthol can also activate central analgesic pathways. At high concentrations, menthol can generate cold allodynia.

Capsaicin can induce an initial pain sensation that is followed by analgesia. Accordingly, capsaicin can reduce itch in psoriatic patients.

Calendula can be used as an antiseptic and anti-inflammatory agent that presents anti-inflammatory and antibacterial activities as well as the capability of stimulating fibroplasia and angiogenesis. Therefore, calendula extracts can act in a positive form on the inflammatory and proliferative phases of the healing process of cutaneous wounds.

Hydrocortisone can share anti-inflammatory, antipruritic and vasoconstrictive actions. Topical corticosteroids can be used for the relief of the inflammatory and pruritic manifestations of corticosteroid-responsive dermatoses.

The hydrocolloid layer 206 with the described properties herein can relieve itches caused by bug bites via an attenuation of the sensor itch pathway, which normally elicits a scratching response. The physical presence and focal pressure of the patch 100 provides a mechanical sensation that is registered by the C fibers in the skin's somatic sensory system. This sensation can compete with pruritogenic stimuli caused by the bug bite and registered by similar C fibers, thereby attenuating the undesirable itch sensation.

Additionally, after scratching, there is damage to the stratum corneum and the impaired barrier function that can induce itch. This leads to the itch-scratch cycle. The hydrocolloid layer 206, however, can protect the epidermis, reducing the amount of scratching, which in turn reduces the duration and after-effects of the bug bite. Open wounds from scratching, which can otherwise lead to infection and/or scarring, are also minimized as a result.

The patch 100 can also protect bug bites from incidental touch and itchiness flare-ups. Uncovered bug bites can easily flare up due to light touch (e.g., from clothes), which can lead to a cycle of itchiness leading to scratching leading to more itchiness. The patch 100, by virtue of having both a backing sheet 202 and a dampening hydrocolloid layer 206, can reduce the sensation caused by incidental touch, and thereby reduces the risk of itchiness flare-ups.

The ingredients included in the hydrocolloid layer 206 as described above can bring their respective benefits to the bug bite. The patch 100 can also act as a wound dressing in case of late application to an already scratched bug bite. The patch 100 can accordingly protect the wound from infection and can allow the epidermis to heal with reduced risk of scarring. Further, the absorptive properties of the hydrocolloid layer 206 can extract some of the venom from the bug bite, thereby reducing the overall venom exposure and associated negative effects such as itchiness.

The patch 100 can comprise a colored hydrocolloid layer 206 or a colored backing sheet 202. The colored opaque patch 100 can provide UPF (ultraviolet protection factor) protection from the sun, preventing hyperpigmentation and scarring. The patch 100 can have a UPF of equal to or greater than 40. Alternatively, said patch 100 can have a UPF from about 15 to about 40.

Both UVA and UVB can lead to increased hyperpigmentation and scarring of the skin. Using UPF protection, this risk is diminished. UPF is a standard used to measure effectiveness of sun protective fabrics or applicators. UPF accounts for both ultraviolet A (UVA) and ultraviolet B (UVB) protection. For example, a UPF of 50 blocks out 49/50 (e.g., 98%) of ultraviolet rays when worn. UPF measurements of fabrics are generally tested by spectrophotometer equipment and are not tested using human subjects. The UPF rate indicates how much of the sun's ultraviolet radiation is absorbed by the fabric. For example, a fabric with a UPF rating of 50 only allows 1/50th of the sun's UV radiation to pass through it. This means that this fabric will reduce your skin's UV radiation exposure by 50 times (98% ultraviolet block) in areas where the skin is protected by the fabric.

The UPF protection of the patch 100 can be increased by providing the backing sheet 202 and/or the hydrocolloid layer 206 with zinc oxide. The zinc oxide added to the backing sheet 202 and/or the hydrocolloid layer 206 can comprise a concentration of about 0.5% to about 2.0% (e.g., about 0.8%).

The UPF protection of the patch 100 can also be increased by increasing the thickness of the backing sheet 202 and/or the hydrocolloid layer 206.

The properties of the patch 100 can improve scar improvement and prevention of hyperpigmentation since after an active bug bite, the epidermal-dermal barrier is disrupted and is therefore left with erythema and an increased risk of hyperpigmentation and scarring. This can result in a faster resolution of the bug bite. Notwithstanding the above, patch 100 can be worn on the skin for as many as 5 days. In other embodiments, the patch 100 can be transparent.

The patch 100 can comprise a thickness of about 0.2 to about 0.3 mm (e.g., about 0.254 mm) and a width of about 11.76 mm to about 17.64 mm.

The patch 100 can comprise a 90° peel adhesion of about 35 oz/in to about 50 oz/in (e.g., about 35.19 oz/in to about 49.98 oz/in). The patch 100 can comprise an absorption percentage of about 131% by weight over 24 hours to about 240% by weight over 24 hours.

Over time, the patch 100 can comprise a tack formulation comprising an absorption percentage of about 380% over 24 hours at 10 months old and about 350% over 24 hours at about 46 months old, signifying a decrease in absorption over time. An adhesion of the patch 100 can be about 60.9 oz/in at 10 months old and about 95.1 oz/in at 46 months old, signifying an increase of adhesion over time. Accordingly, the patch 100 can comprise a shelf life of at least three years.

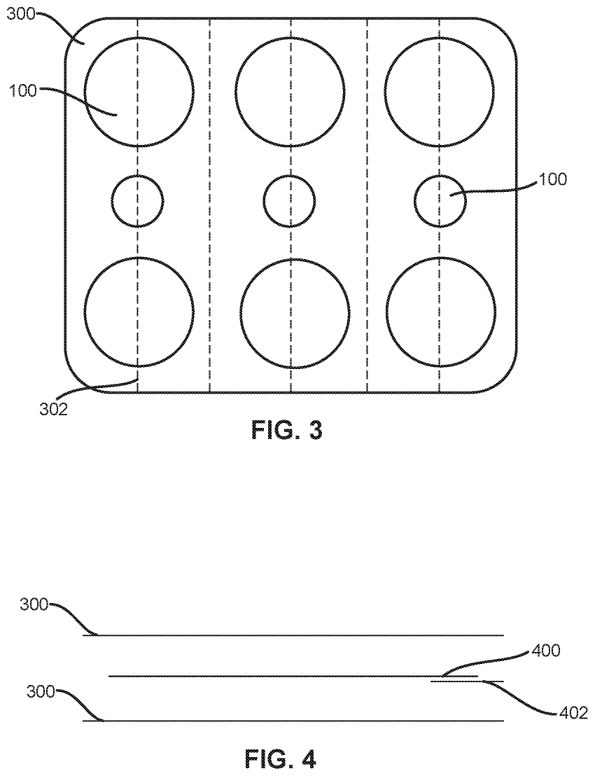

As seen in FIG. 3, patches 100 can be placed on a cold seal paper 300 having latex-free properties. The patches 100 can be placed in rectangular array as shown, though other variations can be used. The patches 100 can comprise different sizes for different sized bug bites or wounds.

The paper 300 can comprise one or more perforations 302 across the width of the paper 300. The perforations 302 can comprise folds within the paper 300 and across each patch 100 to case removal of the patch 100 from the paper 300 for use.

FIG. 4 illustrates a side view of a hydrocolloid sheet 400 comprising patches 100 and placed on a cold seal tab 402 and between two cold seal papers 300. The cold seal tab 402 enables the adhesive of the patch 100 to retain its stickiness during transport and storage while the cold seal papers 300 protect the cold seal tab 402 and the hydrocolloid sheet 400 from any environmental conditions during transport and storage.

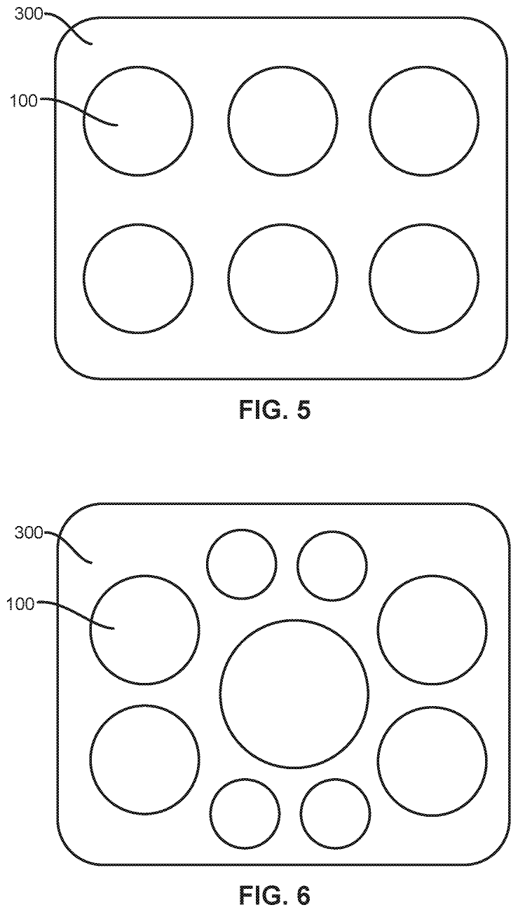

FIGS. 5 and 6 illustrate further variations of one or more patches on a cold seal paper. The patch can have a diameter of about 0.5 in, about 0.75 in, or about 1.00 in. The hydrocolloid sheet 400 can have rounded corners and can have a length of about 2.75 in and a width of about 2.25 in. The patches 100 on the hydrocolloid sheet 400 can be spaced apart about 0.08 in to about 0.09 in from each other and about 0.25 in to about 0.38 in from the edge of the cold seal paper 300.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

It will be understood by one of ordinary skill in the art that the various methods disclosed herein may be embodied in a non-transitory readable medium, machine-readable medium, and/or a machine accessible medium comprising instructions compatible, readable, and/or executable by a processor or server processor of a machine, device, or computing device. The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. A treatment patch for use on a patient's skin, comprising:
a first surface comprising a backing sheet;
a second surface comprising a hydrocolloid layer, wherein the hydrocolloid layer comprises a self-adhesive dressing configured to adhere the treatment patch to the patient at the second surface, wherein the hydrocolloid layer comprises aloe barbadensis leaf and witch hazel embedded within; and
wherein the treatment patch comprises an ultraviolet protection factor (UPF) to improve scarring of the skin.

2. The treatment patch of claim 1, wherein the hydrocolloid layer comprises a thickness of about 12 mm to about 18 mm.

3. The treatment patch of claim 1, wherein the self-adhesive dressing comprises a 90° peel adhesion of about 35.19 oz/in to about 49.98 oz/in.

4. The treatment patch of claim 1, wherein the treatment patch comprises an absorption percentage of about 131% to about 240% by weight over 24 hours.

5. The treatment patch of claim 1, wherein the backing sheet is made of polyurethane.

6. The treatment patch of claim 1, wherein the backing sheet is transparent.

7. The treatment patch of claim 1, wherein the backing sheet is opaque.

8. The treatment patch of claim 1, wherein the treatment patch is configured to adhere to the skin for 5 days.

9. The treatment patch of claim 1, wherein the hydrocolloid layer further comprises menthol.

10. The treatment patch of claim 1, wherein the hydrocolloid layer further comprises capsaicin.

11. The treatment patch of claim 1, wherein the hydrocolloid layer further comprises calendula.

12. The treatment patch of claim 1, wherein the hydrocolloid layer further comprises hydrocortisone.

13. The treatment patch of claim 1, wherein the witch hazel is diluted in water.

14. The treatment patch of claim 1, wherein the aloe barbadensis leaf is purified and chopped into powder.

15. The treatment patch of claim 1, wherein the hydrocolloid layer further comprises zinc oxide configured to increase the ultraviolet protection factor of the treatment patch.

16. A method of using a treatment patch to relieve a region on a patient's skin, the method comprising:
diluting witch hazel in a water solution;
purifying aloe barbadensis leaf and chopping the aloe barbadensis leaf into a powder;
embedding the witch hazel and the aloe barbadensis leaf into a hydrocolloid layer;
providing a treatment patch, wherein the treatment patch comprises: a first surface comprising a backing sheet, a second surface comprising the hydrocolloid layer, wherein the hydrocolloid layer comprises a self-adhesive dressing configured to adhere the treatment patch to the patient at the second surface, and wherein the treatment patch comprises an ultraviolet protection factor (UPF) to improve scarring of the skin; and
administering the treatment patch to the patient's skin.

17. The treatment patch of claim 16, wherein the hydrocolloid layer further comprises menthol.

18. The treatment patch of claim 16, wherein the hydrocolloid layer further comprises capsaicin.

19. The treatment patch of claim 16, wherein the hydrocolloid layer further comprises calendula.

20. The treatment patch of claim 16, wherein the hydrocolloid layer further comprises zinc oxide.
